(19) United States
(12) Patent Application Publication
(10) Pub. No.: US 2007/0120976 A1
(43) Pub. Date: May 31, 2007

(54) METHOD AND DEVICE FOR COMPRESSING IMAGE SIGNAL AND ENDOSCOPE SYSTEM USING SUCH DEVICE

(75) Inventors: Mitsuhiro MATSUMOTO, Tokyo (JP); Koji TSUDA, Tokyo (JP); Tadashi MINAKUCHI, Tokyo (JP); Eiichi ITO, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/563,249

(22) Filed: Nov. 27, 2006

(30) Foreign Application Priority Data
Nov. 28, 2005 (JP) P2005-341758

(51) Int. Cl. A61B 1/04

(52) U.S. Cl. 348/71

(57) ABSTRACT

There is provided a method of compressing an image signal including a plurality of pixel signals outputted from a pixel array in which color pixels are arranged in a predetermined array. The method includes calculating a difference between pixel signals of neighboring same color pixels in the pixel array in accordance with predetermined order where the plurality of pixel signals are outputted from the pixel array, and generating a difference signal representing the calculated difference between the pixel signals of neighboring same color pixels.

### BACKGROUND OF THE INVENTION

The present invention relates to a method and a device for compressing an image signal and an endoscope system using such a device.

Various types of methods and devices for compressing an image signal have become widespread. Examples of such a device are disclosed in Japanese Patent Provisional Publications Nos. 2002-118764 (hereafter, referred to as JP2002-118764A) and 2003-244458 (hereafter, referred to as JP2003-244458A).

Recently, a technology for transmitting a signal (a packet) to a destination via a plurality of DST (Diffusive Signal-Transmission) chips has been proposed as described in Japanese Patent Provisional Publication No. 2003-188882 and on a web site 4“http://www.utri.cojp/venture/venture2.html” (retrieved in November, 2005) by CELLCROSS Co., Ltd (the same contents are also available on the website http://www.cellcross.co.jp/technology.html). Hereafter, such a technology is referred to as a 2D-DST (two-dimensional DST) technology. Each DST chip is formed in a minute size for achieving flexibility of a substrate in which DST chips are provided and reduction in thickness of the substrate. Such a limited size of each DST chip also limits the signal processing performance and the memory size of each DST chip. Therefore, it is difficult to transmit the large amount of data to the destination at a time through the DST chips. It is desirable that the data to be transmitted is compressed and the compressed data is transmitted to the destination via the DST chips.

The technique disclosed in the JP2002-118764A and JP2003-244458A requires relatively high signal processing performance and a relatively large memory size due to its complicated control. Therefore, to achieve the technique disclosed in the JP2002-118764A and JP2003-244458A using the DST chips, each DST chip needs to have relatively high signal processing performance and a relatively large memory size. However, as describe above, it is difficult for each DST chip to have relatively high signal processing performance and relatively large memory size due to its limited chip size. Therefore, it is difficult for each DST chip to have the technique disclosed in JP2002-118764A and JP2003-244458A.

Various image signal compression techniques such as a JPEG (Joint Photographic experts Group) compression technique have been proposed. However, such an image signal compression technique needs to use high processing performance and a frame memory. Therefore, it is difficult for the DST chip to employ such an image signal compression technique,

### SUMMARY OF THE INVENTION

The present invention is advantageous in that at least a method and a device for compressing a signal without requiring relatively high processing performance and a relatively large amount of memory are provided.

According to an aspect of the invention, there is provided a method of compressing an image signal including a plurality of pixel signals outputted from a pixel array in which color pixels are arranged in a predetermined array. The method includes calculating a difference between pixel signals of neighboring same color pixels in the pixel array in accordance with predetermined order where the plurality of pixel signals are outputted from the pixel array, and generating a difference signal representing the calculated difference between the pixel signals of neighboring same color pixels.

With this configuration, it is possible to effectively compress an image signal using a processing circuit having relatively low processing performance and a relatively low memory amount.

In at least one aspect, the calculating the difference is performed for each of the pixel signals outputted from the pixel array line by line.

In at least one aspect, the method further including adding identification information concerning the difference signal to the difference signal.

In at least one aspect, the pixel signals are outputted from the pixel array in the predetermined order such that colors of neighboring pixel signals successively outputted are different from each other

In at least one aspect, the predetermined array includes a Bayer array.

In at least one aspect, the calculating the difference and the generating the difference signal are repeated so that difference signals are generated for all of the plurality of pixel signals of the pixel array,

According to another aspect of the invention, there is provided a method of compressing an image signal including a plurality of pixel signals outputted from a pixel array in which color pixels are arranged in a predetermined array. The method includes separating each of the plurality of pixel signals from the image signal in accordance with color, storing the separated pixel signals in accordance with color, calculating a difference between pixel signals of neighboring same color pixels using the stored pixel signals, and generating a difference signal representing the calculated difference between the pixel signals of neighboring same color pixels.

With this configuration, it is possible to effectively compress an image signal using a processing circuit having relatively low processing performance and a relatively low memory amount.

According to another aspect of the invention, there is provided a method of compressing an image signal including a plurality of pixel signals outputted from a pixel array in which color pixels are arranged in a predetermined array. The method includes separating the plurality of pixel signals from the image signal line by line, storing at least one line of separated pixel signals, calculating a difference between pixel signals in neighboring lines having a same color pixel arrangement using the stored at least one line of separated pixel signals, and generating a difference signal representing the calculated difference between the pixel signals of neighboring same color pixels.

With this configuration, it is possible to effectively compress an image signal using a processing circuit having relatively low processing performance and a relatively low memory amount.

According to another aspect of the invention, there is provided a method of compressing an image signal including a plurality of pixel signals outputted from a pixel array in which color pixels are arranged in a predetermined array. The method includes separating the plurality of pixel signals from the image signal line by line, storing at least one line of separated pixel signals, calculating a difference between pixel signals in a neighboring lines having a same color arrangement using the stored at least one line of separated pixel signals, and generating a difference signal representing the calculated difference if at least one of difference signals calculated for all the pixel signals in the stored at least one line of separated pixel signals is not zero, and generating a notification signal if all the difference signals calculated for all the pixel signals in the stored at least one line of separated pixel signals are zero.

With this configuration, it is possible to effectively compress an image signal using a processing circuit having relatively low processing performance and a relatively low memory amount.

In at least one aspect, the notification signal indicates that all of the difference signals calculated for all the pixel signals in the stored at least one line of separated pixel signals are zero.

According to another aspect of the invention, there is provided a device for compressing an image signal including a plurality of pixel signals outputted from a pixel array in which color pixels are arranged in a predetermined array. The device is provided with a signal obtaining unit configured to obtain the image signal, a signal separation unit configured to separate each of the plurality of pixel signals from the image signal in accordance with color, a plurality of memories respectively storing different color pixel signals separated by the signal separation unit in accordance with color, and a difference signal generation unit configured to calculate a difference between pixel signals of neighboring same color pixels using the pixel signals stored in at least one of the plurality of memories and to generate a difference signal representing the difference.

With this configuration, it is possible to effectively compress an image signal using a processing circuit having relatively low processing performance and a relatively low memory amount.

In at least one aspect, each of the memories is able to store at least two neighboring pixel signals in the image signal.

In at least one aspect, the difference signal generation unit calculates the difference between pixel signals of neighboring same color pixels for all the plurality of pixel signals in the pixel array line by line.

In at least one aspect, the difference signal generation unit adds identification information concerning the difference signal to the difference signal.

In at least one aspect, the pixel array includes a Bayer array. In this case, the plurality of memories may include a first memory storing pixel signals for red pixels in an odd line in the pixel array, a second memory storing pixel signals for green pixels in an odd line in the pixel array, a third memory storing pixel signals for green pixels in an even line in the pixel array, and a fourth memory storing pixel signals for blue pixels in an even line in the pixel array.

In at least one aspect, the pixel array includes a Bayer array. In this case, the plurality of memories may include a first memory storing pixel signals for red pixels in an odd line in the pixel array, a second memory storing pixel signals for green pixels in odd and even lines in the pixel array, and a third memory storing pixel signals for blue pixels in an even line in the pixel array.

According to another aspect of the invention, there is provided a device for compressing an image signal including a plurality of pixel signals outputted from a pixel array in which color pixels are arranged in a predetermined array. The device is provided with a plurality of signal processing units respectively corresponding to different colors of pixels in the pixel array. Each of the plurality of signal processing units includes a signal obtaining unit configured to obtain the image signal, a signal extraction unit configured to extract pixel signals having a predetermined color from the image signal, and a difference signal generation unit configured to calculate a difference between pixel signals of neighboring same color pixels using the pixel signals extracted by the signal extraction unit and to generate a difference signal representing the difference.

With this configuration, it is possible to effectively compress an image signal using a processing circuit having relatively low processing performance and a relatively low memory amount,

According to another aspect of the invention, there is provided a device for compressing an image signal including a plurality of pixel signals outputted from a pixel array in which color pixels are arranged in a predetermined array. The device is provided with a signal obtaining unit configured to obtain the image signal, a signal separation unit configured to separate the plurality of pixel signals from the image signal line by line, a plurality of memories respectively storing different lines of pixel signals separated by the signal separation unit, and a difference signal generation unit configured to calculate a difference between pixel signals in neighboring lines having a same color pixel arrangement using the pixel signals stored in at least one of the plurality of memories and to generate a difference signal representing the difference.

With this configuration, it is possible to effectively compress an image signal using a processing circuit having relatively low processing performance and a relatively low memory amount.

According to another aspect of the invention, there is provided a device for compressing an image signal including a plurality of pixel signals outputted from a pixel array in which color pixels are arranged in a predetermined array. The device is provided with a signal obtaining unit configured to obtain the image signal, a signal separation unit configured to separate the plurality of pixel signals from the image signal line by line, a plurality of memories respectively storing different lines of pixel signals separated by the signal separation unit, a difference calculation unit configured to calculate a difference between pixel signals in neighboring lines having a same color pixel arrangement the pixel signals stored in at least one of the plurality of memories, and a difference signal generation unit configured to generate a difference signal representing the calculated difference if at least one of difference signals calculated for all the pixel signals of a line in the pixel array is not zero, and to generate a notification signal if all difference signals calculated for all the pixel signals in a line in the pixel array are zero.

With this configuration, it is possible to effectively compress an image signal using a processing circuit having relatively low processing performance and a relatively low memory amount.

In at least one aspect, each of the plurality of memories is able to store a line of pixel signals.

In at least one aspect, the pixel array includes a Bayer array. In this case, the plurality of memories includes a first line memory storing pixel signals in an odd line in the pixel array and a second line memory storing pixel signals in an even line in the pixel array.

According to another aspect of the invention, thee is provided a wearable device, which is provided with a substrate including a conductive sheet and a plurality of diffusive signal-transmission chips distributed over the conductive sheet. In this configuration, each of the plurality of diffusive signal-transmission chips comprises one of the above mentioned device. The conductive layer is formed to be able to cover at least a part of a body of a subject, and information on the body of the subject is transmitted through the substrate.

With this configuration, it is possible to effectively compress the image signal using DST chips having relatively low processing performance.

According to another aspect of the invention, there is provided a wearable device, which is provided with a substrate including a conductive sheet and a plurality of diffusive signal-transmission chips distributed over the conductive sheet. In this configuration, at least parts of the plurality of diffusive signal-transmission chips form the above mentioned device including the plurality of signal processing units. The plurality of signal processing units are respectively provided in different ones of the at least parts of the plurality of diffusive signal-transmission chips. The conductive layer is formed to be able to cover at least a part of a body of a subject, and information on the body of the subject is transmitted through the substrate.

With this configuration, it is possible to effectively compress the image signal using DST chips having relatively low processing performance.

According to another aspect of the invention, there is provided an endoscope system, which is provided with a capsule-type endoscope having a form of a capsule, and one of the above mentioned wearable device. The capsule-type endoscope includes an image pickup unit configured to obtain an image of an inside of a body cavity of the subject and to generate an image signal representing the obtained image, and a wireless communication unit configured to transmit the image signal as a radio signal. The signal obtaining unit in the wearable device includes an antenna which receives the image signal transmitted from the wireless communication unit of the capsule-type endoscope.

With this configuration, it is possible to effectively compress the image signal using DST chips having relatively low processing performance,

### BRIEF DESCRIPTION OF THE ACCOMPANYING DRAWINGS

FIG. 1 is a block diagram of an endoscope system according to a first embodiment of the invention.

FIG. 2 is a block diagram of a capsule-type endoscope provided in the endoscope system.

FIG. 3 illustrates a color filter on which primary color filters are arranged in a so-called Bayer array.

FIG. 4 is a cross section of a diagnostic jacket illustrating a structure of layers of the diagnostic jacket,

FIG. 5 is a block diagram of a DST chip according to the first embodiment.

FIG. 6 is a flowchart illustrating an image signal compression process executed by the DST chip according to the first embodiment.

FIG. 7 shows an image signal converted by an A-D converter in the DST chip.

FIG. 8 is a flowchart illustrating a signal separation process executed in the image signal compression process.

FIGS. 9A to 9F are explanatory illustrations for explaining effect of a difference operation process executed in the image signal compression process,

FIG. 10 is a block diagram of an endoscope system according to a second embodiment of the invention.

FIG. 11 is a block diagram of a DST chip according to the second embodiment.

FIG. 12 is a flowchart illustrating an image signal compression process executed by the DST chip according to the second embodiment.

FIG. 13 is a flowchart illustrating a signal extraction process executed in the image signal compression process shown in FIG. 12.

FIG. 14 is a block diagram of a DST chip according to the third embodiment.

FIGS. 15A to 15C are explanatory illustrations for explaining effect of a difference operation process executed in an image signal compression process according to a third embodiment.

FIG. 16 is a flowchart illustrating the image signal compression process executed by the DST chip according to the third embodiment.

FIG. 17 is a block diagram of a DST chip according to a fourth embodiment.

FIG. 18 is a flowchart illustrating the image signal compression process executed by the DST chip according to the fourth embodiment.

FIGS. 19A to 19C are explanatory illustrations for explaining the case where output values of pixel signals in targeted two lines are equal to each other.

### DETAILED DESCRIPTION OF THE EMBODIMENTS

Hereinafter, embodiments according to the invention are described with reference to the accompanying drawings.

### First Embodiment

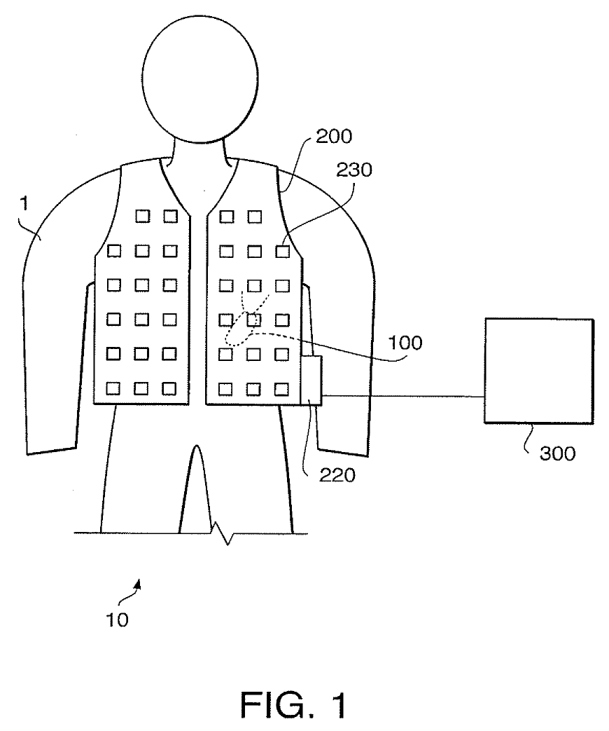

FIG. 1 is a block diagram of an endoscope system 10 according to a first embodiment of the invention. The endoscope system 10 is used to observe the inside of a body cavity of a subject 1 for medical diagnostic purpose. As shown in FIG. 1, the endoscope system 10 includes a capsule-type endoscope 100, a diagnostic jacket (i.e., a wearable device) 200, and a PC (personal computer) 300 having a monitor. The capsule-type endoscope 100 is formed to be a small size endoscope capable of getting into the inside of a body cavity for imaging the inside of the body cavity.

The diagnostic jacket 200 is worn by the subject 1 so that image data output by the capsule-type endoscope 100 can be obtained. On the monitor of the PC 300, observation images can be displayed.

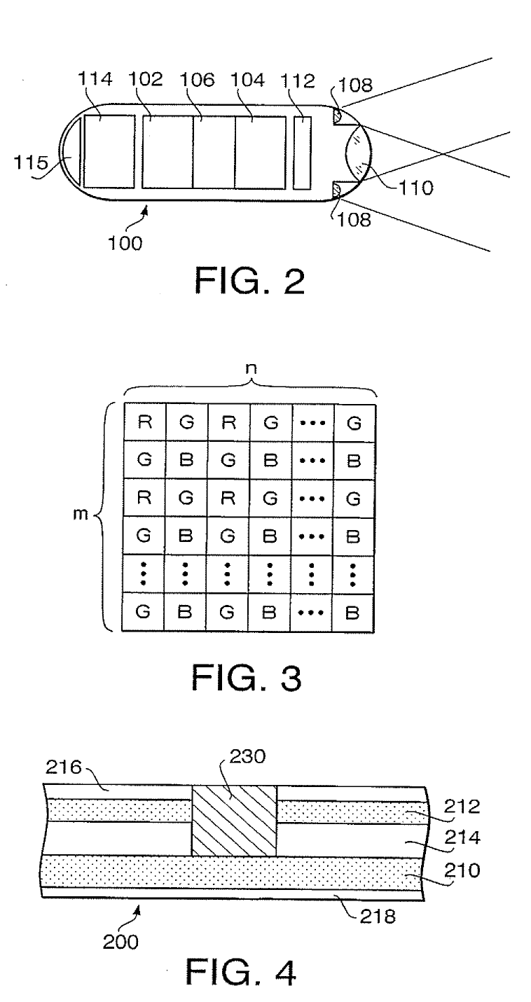

FIG. 2 is a block diagram of the capsule-type endoscope 100. Since the capsule-type endoscope 100 is small and is formed in a shape of a capsule, the capsule-type endoscope 100 is able to get into an inside of a meandering narrow long tube (e.g., a bowel) and to image the inside of the tube. As shown in FIG. 2, the capsule-type endoscope 100 includes a power supply unit 102, a control unit 104 for controlling the capsule-type endoscope 100, a memory 106 in which various types of data is stored, an illumination units 108 for illuminating the inside of a body cavity, an objective optical system 110 for forming an image of the inside of a body cavity, a solid-state image pickup device 112, and an output unit 114 for outputting a radio wave, and an antenna 115 through which a radio wave is transmitted or received.

When power of the capsule-type endoscope 100 is turned to ON and the capsule-type endoscope 100 is inserted into a body cavity, the capsule-type endoscope 100 illuminates the inside of the body cavity with the illumination units 108. Light reflected from an inside wall of the body cavity enters the objective optical system 110, and the objective optical system 100 forms an image on an image side focal plane (i.e., a light reception surface of the solid-state image pickup device 112).

The solid-state image pickup device 112 which receives the image formed by the objective optical system 110 has n by m pixels arranged in a matrix (n pixels in a horizontal direction and m pixels in a vertical direction) and is able to generate color images. The solid-state image pickup device 112 has a color filter on the front side of the light reception surface thereof. FIG. 3 illustrates the color filter on which primary color filters (i.e., R(red), G(green) and B(blue) filters) are arranged in a so-called Bayer array. More specifically, on each odd line, R and G filters are alternately arranged (i.e., arranged in a form of R, G, R, G . . . ). On each even line, G and B filters are alternately arranged (i.e., arranged in a form of G, B, G, B, . . . ).

The solid-state image pickup device 112 converts the image formed thereon to an electronic image signal. The control unit 104 controls the output unit 114 to modulate the image signal generated by the solid-state image pickup device 112. Then, the modulated image signal is transmitted through the antenna 115 as a radio signal. The radio signal (i.e., an analog image signal) outputted by the capsule-type endoscope 100 is received by the diagnostic jacket 200.

A configuration and operations of the diagnostic jacket 200 will now be described. As shown in FIG. 1, the diagnostic jacket 200 is formed to cover a part of a body of the subject 1. A plurality of DST chips 230 each of which is configured to transmit a signal based on the 2D-DST technology are distributed over the diagnostic jacket 200. In the diagnostic jacket 200, a transmission channel for transmitting the image signal outputted by the capsule-type endoscope 100 can be formed without using a metal pattern or a wired line. By employing the 2D-DST technology, the diagnostic jacket 200 is able to achieve substantially the same flexibility and durability as clothing. Such a configuration of the diagnostic jacket 200 makes it possible to configure signal receiving chips communicating with the capsule-type endoscope 200 in a high degree of freedom and in a high density. The diagnostic jacket 200 includes a control unit 220 configured to control the entire circuit of the diagnostic jacket 200.

FIG. 4 is a cross section of the diagnostic jacket 200 illustrating a structure of layers of the diagnostic jacket 200. As shown in FIG. 4, the diagnostic jacket 200 has a laminated structure of two conductive sheets 210 and 212, an insulating sheet 214 providing electrical insulation between the two conductive sheets 210 and 212, and insulating sheets 216 and 218 for insulating each conductive sheet from the outside. In this laminated structure, the insulating sheet 216 is situated on the subject side, and the insulating sheet 218 is situated on the outside of the diagnostic jacket 200. Each DST chip 230 is imbedded in the laminated structure across the insulating sheet 216, the conductive sheet 212 and the insulating sheet 214. The DST chips are distributed over the entire region of the diagnostic jacket 200.

Each of the conductive sheets 210 and 212 has flexibility and conductivity, and is formed to surround the body of the subject from a chest region to an abdominal region. Each of the conductive sheets 210 and 212 is made of a conductive rubber or fabric into which a conductive material is incorporated. The conductive sheet 210 is set at a ground level The conductive sheet 212 functions as a signal layer through which a signal (i.e., an image signal from the capsule-type endoscope 100) is transmitted between the DST chips 230 in accordance with the 2D-DST technology.

Each of the insulating sheets 214, 216 and 218 has flexibility and an insulating property, and is formed of, for example, insulating rubber, an insulating film, or fabric having an insulating property. The insulating sheet 214 is sandwiched between the conductive sheets 210 and 212 to provide electrical insulation between the conductive sheets 210 and 212. The insulating sheet 216 is formed to cover the outer surface of the conductive sheet 212, The insulating sheet 218 is formed to cover the outer surface of the conductive sheet 210. By an insulating property of each of the insulating sheets 214, 216 and 218, electrical insulation between the conductive layers can be maintained and electrical insulation between each conductive sheet (210,212) and the outside (e.g., a surface of the body of the subject 1) can be maintained.

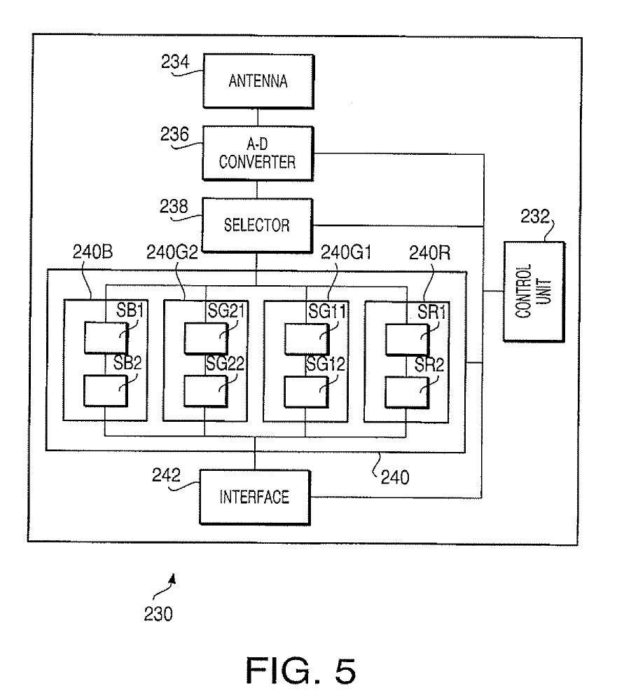

FIG. 5 is a block diagram of the DST chip 230 according to the first embodiment. As shown in FIG. 5, the DST chip 230 includes a control unit 232, an antenna 234, an A-D converter 236, a selector 238, a storage unit 240 and an interface 242. The storage unit 240 includes four memories 240R, 24001, 240G2 and 240B.

In order to set up the DST chips to receive the image signal from the capsule-type endoscope 100, the control unit 220 operates to compare signal reception levels of signals received by antennas 234 of all the DST chips 230 in the diagnostic jacket 200. Then, the control unit 220 selects a DST chip 230 of which antenna 234 has the highest signal reception level, and sets the DST chip 230 having the highest signal reception level as a signal reception chip. The DST chip 230 set as the signal reception chip operates to receive the image signal transmitted by the capsule-type endoscope 100. The diagnostic jacket 200 thus moves to a state of being able to catch the image signal from the capsule-type endoscope 100. Since reception conditions of the image signal from the capsule-type endoscope 100 changes with time, the control unit 220 may operate to conduct the comparison of signal reception levels periodically to select a DST chip having the maximum signal reception level.

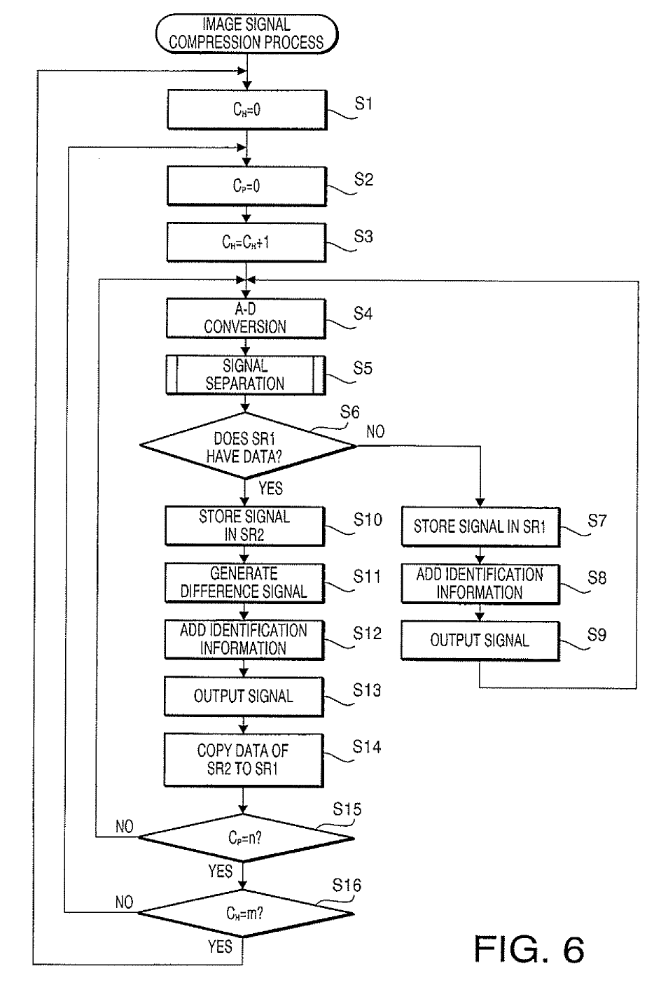

When the image signal is received by the antenna 234 of the signal reception chip, the control unit 232 executes an image signal compression process for compressing the image signal from the capsule-type endoscope 100. FIG. 6 is a flowchart illustrating the image signal compression process executed under control of the control unit 232 of the DST chip 232 (the signal reception chip). The image signal compression process terminates when power of the diagnostic jacket 200 is turned to off or when the diagnostic jacket 200 moves to a state where no image signal is received.

The control unit 232 has a counter (i.e., a counting function) for counting the number of horizontal synchronization signals H and pixels (pixel signals) contained in the image signal. When the control unit 232 reads a vertical synchronization signal V located at a header part of the image signal, the control unit 232 resets the counter (i.e., the count C_{H} of the number of horizontal synchronization signals and the count C_{P} of the number of pixels) to zero (steps S1 and S2). Then, the control unit 232 reads the horizontal synchronization signal and increments the count C_{H} (step S3).

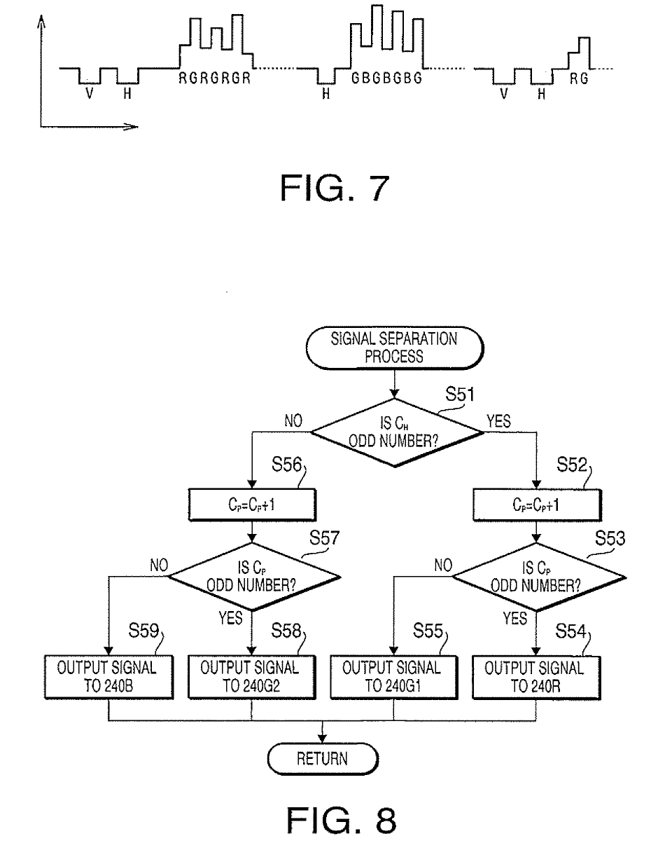

When the image signal received by the antenna 234 is inputted to the A-D converter 236, the control unit 232 controls the A-D converter 236 to convert the analog image signal to a digital signal (i.e., a signal representing digital information) (step S4). FIG. 7 shows an example of an image signal (i.e., a digital signal) converted by the A-D converter 236. In FIG. 7, a vertical axis represents an output level of the image signal, and a horizontal axis represents time. The image signal shown in FIG. 7 is inputted to the selector 238 in chronological order as shown in FIG. 7,

Next, the control unit 232 executes a signal separation process to separate each pixel signal from the image signal (step S5). FIG. 8 is a flowchart illustrating the signal separation process.

When the separation process is initiated, the control unit 232 judges whether the count C_{H} representing the number of horizontal synchronization signals has an odd number (step S51). If the count C_{H} has an odd number (S51: YES), the control unit 232 executes signal separation for the pixels arranged along an odd line in the pixel array on the light reception surface of the solid-state image pickup device 112. More specifically, in step S52, the control unit 232 reads a pixel signal on an odd line in the pixel array, and increments the count C_{P} by one. Then, the control unit 232 judges whether the count C_{P} has an odd number (step S53). If the count C_{P} has an odd number (S53: YES), the control unit 232 judges that the pixel signal represents an R (red) pixel and controls the selector 238 to output the pixel signal to the memory 240R of the storage unit 240 (step S54). If the count C_{P} does not have an odd number (S53: NO), the control unit 232 judges that the pixel signal represents a G (green) pixel and controls the selector 238 to output the pixel signal to the memory 240G1 of the storage unit 240 (step S55).

If it is judged in step S51 that the count C_{H} does not have an odd number (S51: NO), the control unit 232 executes the signal separation for the pixels arranged along an even line in the pixel array on the light reception surface of the solid-state image pickup device 112. More specifically, in step S56, the control unit 232 reads a pixel signal on an even line in the pixel array, and increments the count C_{P} by one. Then, the control unit 232 judges whether the count C_{P} has an odd number (step S57). If the count C_{P} has an odd number (S57: YES), the control unit 232 judges that the pixel signal represents a G (green) pixel and controls the selector 238 to output the pixel signal to the memory 240G2 of the storage unit 240 (step S58). If the count C_{P} does not have an odd number (S57: NO), the control unit 232 judges that the pixel signal represents a B (blue) pixel and controls the selector 238 to output the pixel signal to the memory 240B of the storage unit 240 (step S59).

The pixel signals are thus separated from the image signal and stored in the storage unit 240. That is, the pixel signals of R-pixels arranged along an odd line of the pixel array on the light reception unit of the solid-state image pickup device 112 are stored in the memory 240R. The pixel signals of G-pixels arranged along an odd line of the pixel array are stored in the memory 240G1. The pixel signals of G-pixels arranged along an even line of the pixel array are stored in the memory 240G2. The pixel signals of B-pixels arranged along an even line of the pixel array are stored in the memory 240B.

Each of the memories 240R, 240G1, 240G2 and 240B has two memory segments. Specifically, the memory 240R has memory segments SR1 and SR2. Each of the memory segments SR1 and SR2 holds data only when power is supplied. Therefore, in an initial state, each memory segment does not hold data. The memory 240G1 has memory segments SG11 and SG12, the memory 240G2 has memory segments SG21 and S022, and the memory 240B has memory segments SB1 and SB2. Since these memory segments SG11, SG12, SG21, SG22, SB1 and SB2 have the same configuration as those of the memory segments SR1 and SR2, explanations thereof will not be repeated.

In the following explanations on the image signal compression process shown in FIG. 6, it is assumed that an R-pixel signal is output by the selector 238. Referring back to FIG. 6, when the pixel signal (the R-pixel signal) separated by the selector 238 is outputted, the control unit 232 judges whether memory segment SR1 of the memory 240 holds data (step S6). If the segment SR1 does not hold data (S6: NO), the control unit 232 stores data of the R-pixel signal in the memory segment SR1 (step S7). In this case, the control unit 232 uses one of memory segments in order of precedence of the vacant memory segment, the memory segment SR1, and the memory segment SR2 to store the R-pixel signal in the memory 240R. After step S7 is executed, the memory 240R is in a state where only the memory segment SR1 is filled with the R-pixel signal.

The R-pixel signal stored in step S7 is a first R-pixel signal on each odd line on the pixel array as described below in a difference operation process. This first R-pixel signal is used as a reference signal for the difference operation process. Therefore, the control unit 232 assigns an identification (the count C_{H} of the horizontal synchronization signals and the count C_{P} of the pixels) to the R-pixel signal in a state where the pixel signal is not processed (step S8). Then, the control unit 232 outputs the pixel signal to the interface 242 (step S9). Then, control returns to step S4 to execute the signal separation process again.

If the segment SR1 holds data (S6: YES), the control unit 232 stores the R-pixel signal in the memory segment SR2 (step S10). When the R-pixel signal is thus stored in the memory segment SR2, the memory 240R is in a state where all of the memory segments SR1 and SR2 are filled with data. It should be note that even if the memory segment SR2 is filled with data, the control unit 232 overwrites the memory segment SR2 with the R-pixel signal in step S10.

Next, in step S11, the control unit 232 refers to the data of the R-pixel signals stored in the memory segments SR1 and SR2 to calculate a difference between the output values of the R-pixel signals in the memory segments SR1 and SR2 and thereby to generate a difference signal representing the calculated difference (step S11). The difference is obtained by subtracting the value of the signal output of the memory segment SR2 from the value of the signal output of the memory segment SR1. It is understood that the values of the memory segments SR1 and SR2 correspond to the neighboring same color pixels (i.e., the R-pixels between which a G-pixel signal lies) on the pixel array on the light reception surface of the solid-state image pickup device 112. Therefore, these same color signals have strong correlation with respect to each other. In other words, the output value of the difference signal takes a small value. As a result, the R-pixel signal can be compressed at a high compression rate.

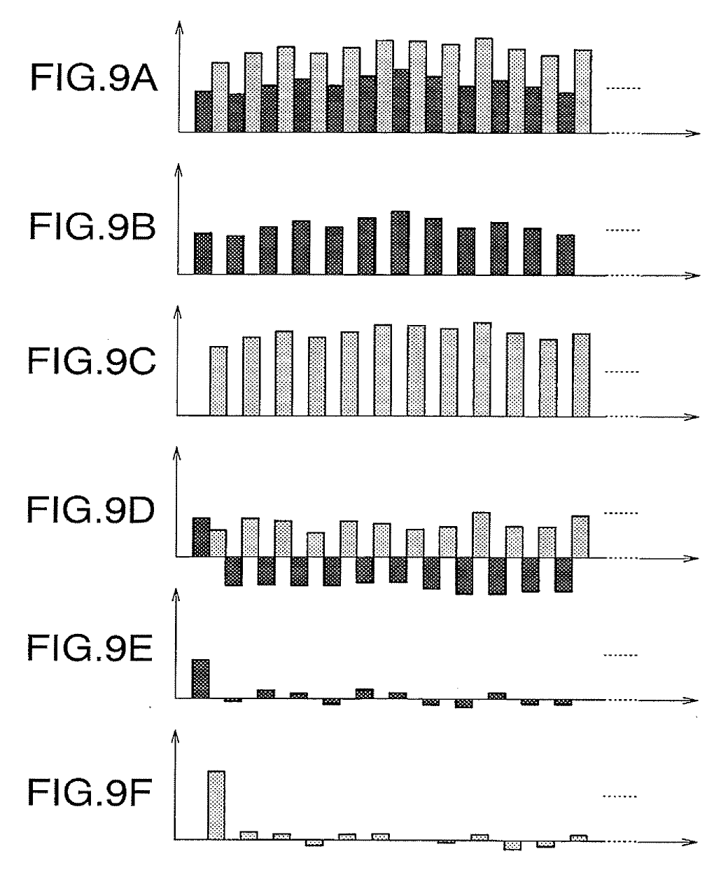

FIGS. 9A to 9F illustrate effect of the difference operation process. FIG. 9A represents output values of all the pixels on an odd line of the pixel array. FIG. 9B represents output values of R-pixels on the odd line shown in FIG. 9A. FIG. 9C represents output values of G-pixels on the odd line shown in FIG. 9A. FIG. 9D represents output values of difference signals between neighboring pixels on the odd line. FIG. 9E represents output values of difference signals between neighboring R-pixels on the odd line. FIG. 9F represents output values of difference signals between neighboring G-pixels on the odd line. In each of FIGS. 9A to 9F, a vertical axis represents an output level of the pixel signal and a horizontal line represents pixels on an odd line on the pixel array.

As shown in FIG. 9A, output values of neighboring R and G pixels on an odd line have low correlation because the neighboring R and G pixels are different in color with respect to each other. Therefore, as shown in FIG. 9D, each difference signal has a relatively high output value. That is, in this case, the image signal is not effectively compressed.

On the other hand, if a difference signal is calculated from neighboring same color pixels, an output value of the difference signal takes a low value because the neighboring same color pixels have strong correlation with each other, as indicated in FIGS. 9E and 9F. That is, in this case, the image signal is effectively compressed.

After obtaining the difference signal, the control unit 232 assigns the identification of the R-pixel signal currently stored in the memory segment SR2 to the difference signal (step S12), and outputs the difference signal to the interface 242 (step S13). Then, the control unit 232 copies the R-signal currently stored in the memory segment 8R2 to the memory segment SR1 (step S14).

Next, in step S15, the control unit 232 judges whether the count C_{P} of the pixel signal is equal to n. If the count C_{P} of the pixel signal is not equal to n (S15: NO), the control unit 232 judges that a sequence of steps for all the pixels on a current line is not finished, and control returns to step S4 to execute the signal separation for another pixel signal on the current line. If the count C_{P} of the pixel signal is equal to n (S15: YES), the control unit 232 judges that a sequence of steps for all the pixels on the current line is finished, and control proceeds to step S16.

In step 516, the control unit 232 judges whether the count C_{H} of the horizontal synchronization signal is equal to m. If the count C_{H} of the horizontal synchronization signal is equal to m (S16: YES), the control unit 232 judges that a sequence of steps for a current frame is finished, and control returns to step S1 to execute the signal separation for a next frame. If the count C_{H} of the horizontal synchronization signal is not equal to m (S16: NO), the control unit 232 judges that a sequence of steps for the current frame is not finished, and control returns to step S2 to execute the signal separation for a next line.

The interface 242 is electrically connected to the conductive sheets 210 and 212. Each pixel signal provided to the interface 242 in step S9 or S13 is transmitted to the control unit 220 while passing through the signal layer (the conductive sheet 212) and being relayed by the appropriately selected DST chips 230 which have been selected as the transmission channel. The pixel signals are inputted to the control unit 220 in the order where the pixels are arranged in the pixel array (“R, G, R, G, . . . ”).

Then, the control unit 220 decompresses each pixel signal (i.e., obtains the output value of each pixel signal in a state before execution of the difference operation process) in accordance with the reference signal and difference signals of each line. The control unit 220 refers to the identification of each pixel signal and stores a frame of decompressed pixel signals, for example, in a memory provided therein. When a frame of decompressed pixel signals is stored, the control unit 220 subjects the pixel signals to image processing to output a frame of image to the PC 300 with the monitor so that an image of the inside of the body cavity of the subject 1 captured by the capsule-type endoscope 100 is displayed on the monitor of the PC 300.

In the above explanations on the image signal compression process shown in FIG. 6, the image signal compression for R-pixel signals are treated for the sake of simplicity. It is understood that G-pixel signals in an odd line, G-pixel signals in an even line, and B-pixel signals are also compressed in the image signal compression process in the same fashion.

As described above, by generating the difference signal using neighboring same color pixels, the image signal can be compressed at a high compression rate through use of a relatively simple circuit.

### Second Embodiment

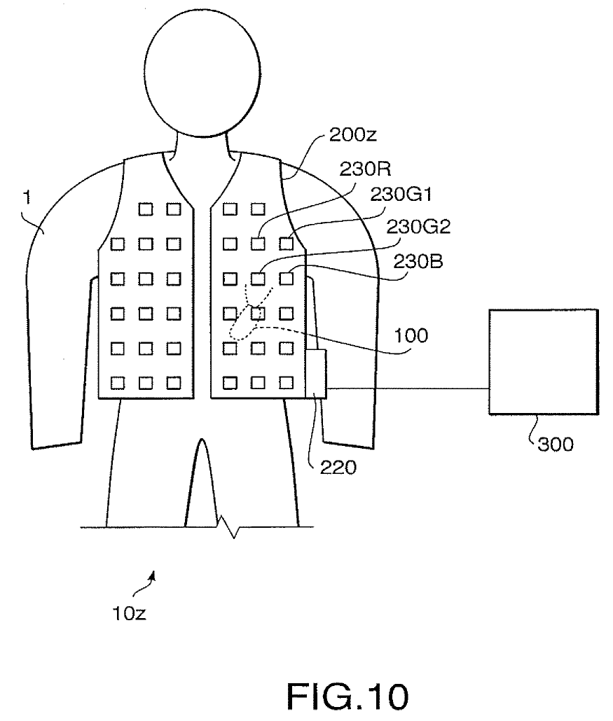

FIG. 10 is a block diagram of an endoscope system 10z according to a second embodiment of the invention. In FIG. 10, to elements, which are substantially the same as those of the first embodiment, the same reference numbers are assigned, and explanations thereof will not be repeated, As shown in FIG. 10, the endoscope system 10z includes the capsule-type endoscope 100, a diagnostic jacket 200z, and the PC 300 with the monitor. The diagnostic jacket 200z includes four types of DST chips (DST chips 230R, 230G1, 230G2 and 230B). The DST chips are uniformly distributed over the entire region of the diagnostic jacket 200z.

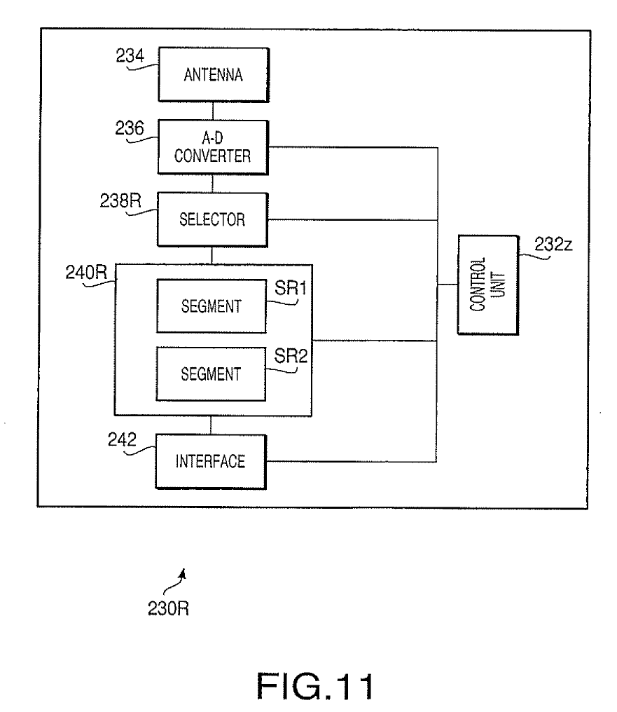

FIG. 11 is a block diagram of the DST chip 230R. As shown in FIG. 11, the DST chip 230R includes a control unit 232z, the antenna 234, the A-D converter 236, a selector 238R, the memory 240R and the interface 242. The memory 240R includes memory segments SR1 and SR2.

Under control of the control unit 220, signal reception levels of all of the DST chips in the diagnostic jacket 200z are compared so that a DST chip having the maximum signal reception level can be selected. The control unit 220 selects the DST chip having the maximum signal reception level as a signal reception chip. In this embodiment, if the DST chip 230R is selected as a signal reception chip, the control unit 220 also selects the DST chips 23001, 230G2 and 230B adjoining to the DST chip 230R as signal reception chips. If the DST chip 230G2 is selected as a signal reception chip, the control nit 220 may select the DST chips 230R, 23001, and 230B adjoining to the DST chip 230G2 as signal reception chips. That is, four types of DST chips 230R, 230G1, 230G2 and 230B are selected as signal reception chips. The antenna 234 of each DST chip selected as a signal reception chip operates to catch the image signal transmitted from the capsule-type endoscope 100.

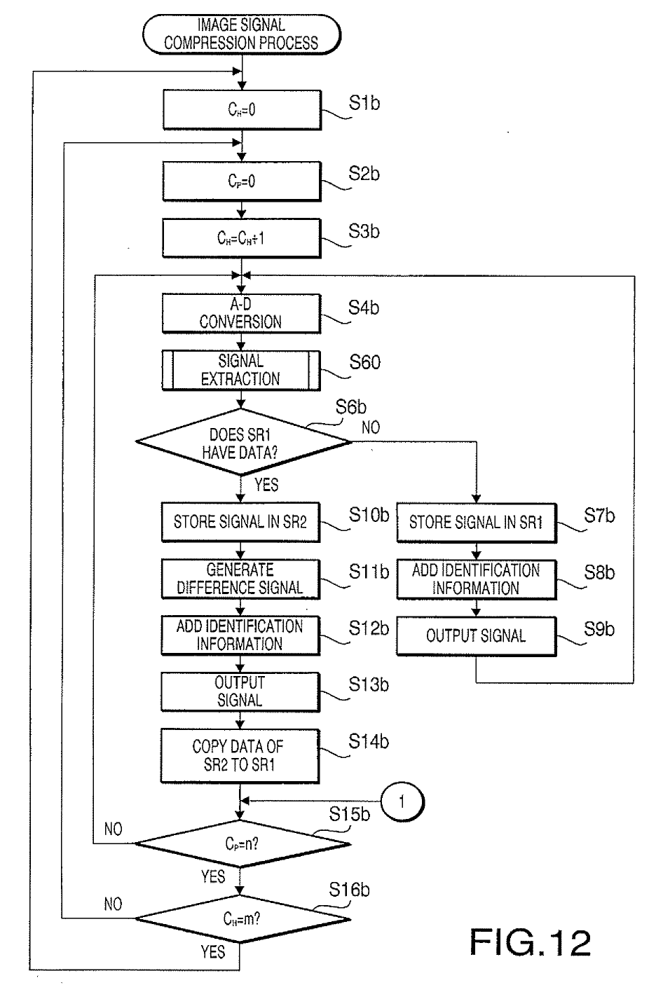

FIG. 12 is a flowchart illustrating an image signal compression process executed under control of the control unit 232z of the DST chip 230R. Since each of the DST chips 23001, 230G2 and 230B has the same configuration and functions as those of the DST chip 230R, explanations thereof will not be repeated.

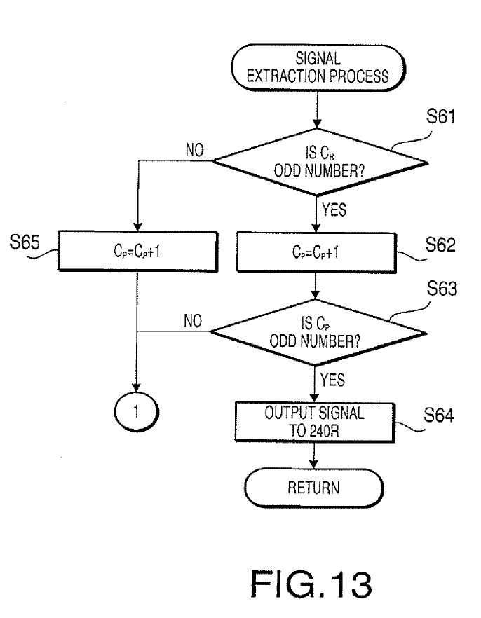

The control unit 232z executes steps S1b to S4b which are substantially the same as steps S1 to S4 in FIG. 6. Then, a signal extraction process is executed in step S60. FIG. 13 is a flowchart illustrating the signal extraction process. In the signal extraction process, only pixel signals satisfying a predetermined condition are extracted and outputted to the memory. With regard to the DST chip 230R, the predetermined condition is a condition for selecting only R-pixel signals. With regard to the DST chip 230G1, the predetermined condition is a condition for selecting only G-pixel signals on each odd line in the pixel array. With regard to the DST chip 230G2, the predetermined condition is a condition for selecting only G-pixel signals on each even line in the pixel array. With regard to the DST chip 2308, the predetermined condition is a condition for selecting only B-pixel signals.

In the signal extraction process, the control unit 232z judges whether the count C_{H} representing the number of horizontal synchronization signals has an odd number (step S61). If the count C_{H} has an odd number (S61: YES), the control unit 232z executes signal extraction for the pixels arranged along an odd line in the pixel array on the light reception surface of the solid-state image pickup device 112. More specifically, in step S62, the control unit 232z reads a pixel signal on an odd line in the pixel array, and increments the count C_{P} by one. Then, the control unit 232z judges whether the count C_{P} has an odd number (step S63). If the count C_{P} has an odd number (S63: YES), the control unit 232z judges that the current pixel signal is an R-pixel signal and controls the selector 238 to output the signal to the memory 240R (step S64).

If the count C_{P} does not have an odd number (S63: NO), the control unit 232z judges that the pixel signal is not an R-pixel signal and controls the selector 238 not to output the pixel signal to the memory 240R. Then, control proceeds to step S15 of FIG. 12.

If it is judged in step S61 that the count C_{H} does not have an odd number (S61: NO), the control unit 232z reads a pixel signal on an odd line in the pixel array, and increments the count C_{P} by one (step S65). In this case, the control unit 232z judges that the pixel signal inputted to the selector 238 is a pixel signal on an even line in the pixel array (i.e., the pixel signal is not an R-pixel signal) and controls the selector 238 not to output the signal to the memory 240R. Then, control proceeds to step S15 of FIG. 12.

When the R-pixel signal is output by the selector 238 in step S64, the control unit 232z stores the R-pixel signal in one of the memory segments in the memory 240R in accordance with the status of the memory segments, and executes the difference operation for the pixel signals stored in the memory segments to output a result of the difference operation through the interface 242 as shown in steps S7b to S9b (which are substantially the same as steps S7 to S9 in FIG. 6) or S10b to S14b (which are the same as steps S10 to S14 in FIG. 6). In response to the statuses of the count C_{H} and count C_{P}, the image signal compression process is repeated as shown in steps S15b and S16b which are substantially the same as steps S15 and S16 in FIG. 6.

Since the DST chip 230R needs to output only R-pixel signals, the DST chip 230 is required to have only the memory 240R. That is, a memory size in each DST chip can be reduced. Such an advantage is also applied to other DST chips (230G1, 230G2, 230B). Since, according to the second embodiment, the memory size in each DST chip can be reduced in comparison with the DST chip in the first embodiment, and cost reduction can be achieved.

The pixel signals of the four DST chips selected as the signal reception chips are sequentially subjected to the image signal compression process in the order of the pixel arrangement in the Bayer array. Then, the pixel signals are transmitted to the control unit 220 while passing through the conductive sheet 210 and being relayed by the DST chips selected to form the signal transmission channel in accordance with the 2D-DST technology. More specifically, the reference signal of the R-pixel signal compressed in the DST chip 230R is transmitted, and then the reference signal of the G-pixel signal is transmitted. Subsequently, the difference signal of the neighboring R-pixel and the difference signal of the neighboring G-pixel signal are transmitted repeatedly as shown in FIG. 7. As a result, the R and G pixel signals are inputted to the control unit 220 as shown in FIG. 7. When a target line to be processed is changed, the reference signal of the G-pixel signal is transmitted and then the reference signal of the B-pixel signal is transmitted. Subsequently, the difference signal of the neighboring G-pixel and the difference signal of the neighboring B-pixel signal are transmitted repeatedly. As a result, the G and B pixel signals are inputted to the control unit 220.

The control unit 220 decompresses each pixel signal (i.e., obtains the output value of each pixel signal in the state before execution of the difference operation process) in accordance with the reference signal and difference signals of each line. When a frame of decompressed pixel signals is stored, the control unit 220 subjects the pixel signals to image processing to output a frame of image to the PC 300 with the monitor so that an image of the inside of the body cavity of the subject 1 captured by the capsule-type endoscope 100 is displayed on the monitor of the PC 300.

### Third Embodiment

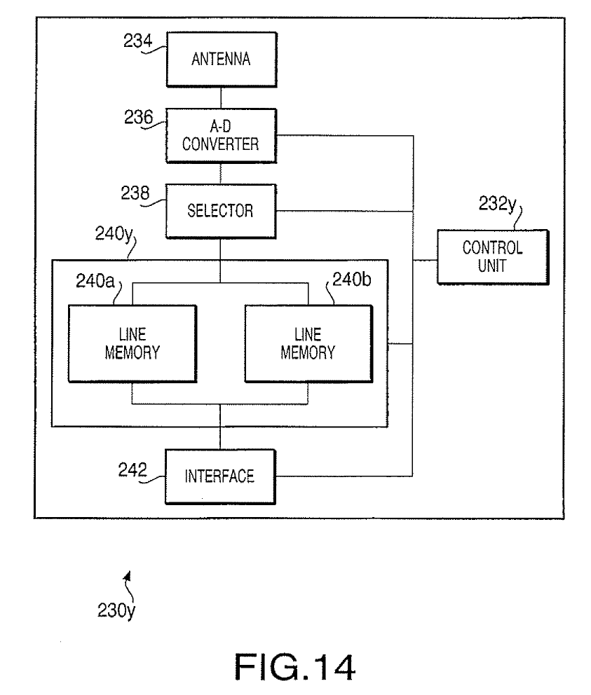

Hereafter, an endoscope system according to a third embodiment is described. Since a general configuration of the endoscope system according to the third embodiment is substantially the same as that of the first embodiment, the drawings of the first embodiment are also used to explain the endoscope system according to the third embodiment. In this embodiment, DST chips 230y (see FIG. 14) are distributed over the entire region of the diagnostic jacket 200. In this embodiment, to elements, which are substantially the same as those of the first embodiment, the same reference numbers are assigned, and explanations thereof will not be repeated.

FIG. 14 is a block diagram of a DST chip 230y according to the third embodiment. As shown in FIG. 14, the DST chip 230y includes a control unit 232y, the antenna 234, the A-D converter 236, the selector 238, the memory 240Y, and the interface 242. The memory 240Y includes line memories 240a and 240b. Each of the line memories 240a and 240b has a memory size enough to store a line of pixel signals. The line memory 240a stores pixel signals in each odd line in the pixel array, and the line memory 240b stores pixel signals in each even line in the pixel array.

Although in this embodiment two different line memories are provided in the DST chip 230y, the DST chip 230y may be configured to have a single line memory. If the DST chip 230y is configured to have a single line memory, the selector 238 operates under control of the control unit 232y to assign addresses to pixel signals such that a pixel signal at a column address on a line in the pixel array (i.e., to be stored in the line memory 240a) and a pixel signal at the same column address on another line in the pixel array (i.e., pixel signals to be stored in the line memory 240b) have different addresses. In this case, the DST chip having the single line memory operates as if the DST chip has separate memory areas corresponding to the line memories 240a and 240b.

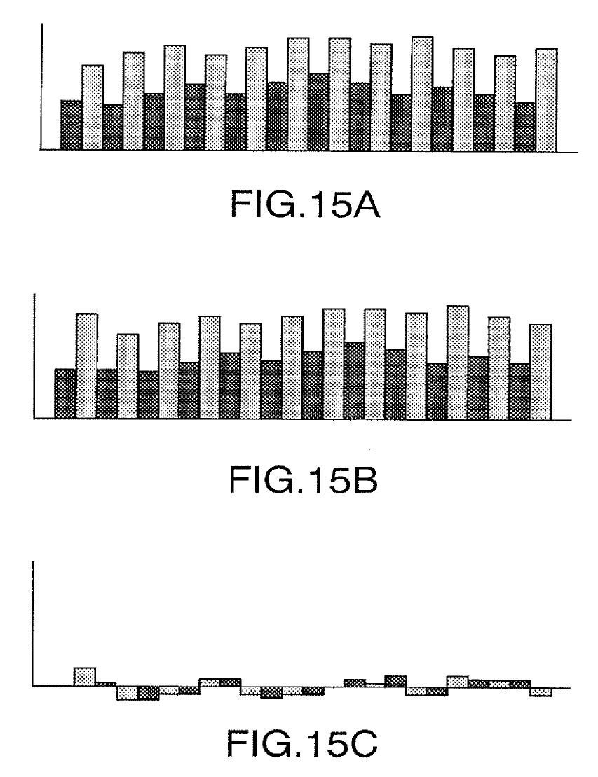

FIGS. 15A to 15C are explanatory illustrations for explaining the effect of a difference operation process according to the third embodiment. FIG. 15A illustrates output values of pixel signals in an (i-2)-th line (where i is a natural number larger than or equal to 3) in the pixel array. FIG. 15B illustrates output values of pixel signals in an i-th line. FIG. 15C illustrates output values of difference signals representing differences between output values of an (i-2)-th line and output values of an i-th line in the pixel array. In FIGS. 15A to 15C, the vertical line represents an output level of a pixel signal, and the horizontal line represents pixels arranged in a horizontal direction on the solid-state image pickup device 112.

On the pixel array of the solid-state image pickup device 112, an (i-1)-th line and an i-th line have different arrangements of color pixels. That is, one of these neighboring lines has the arrangement of “R, G, R, G . . . ”, while the other has the arrangement of “G, B. G, B . . . ”. Therefore, these lines have low correlation with each other although these lines are adjacent to each other in the pixel array. That is, in this case, the image signal is not effectively compressed.

On the other hand, if the difference operation process is applied to lines having the same arrangement of color pixels after the signal separation is performed for each line, the output values of difference signals become low levels because in this case the pixels in the lines have strong correlation with each other as shown in FIG. 15C. That is, in this case, the image signal is effectively compressed.

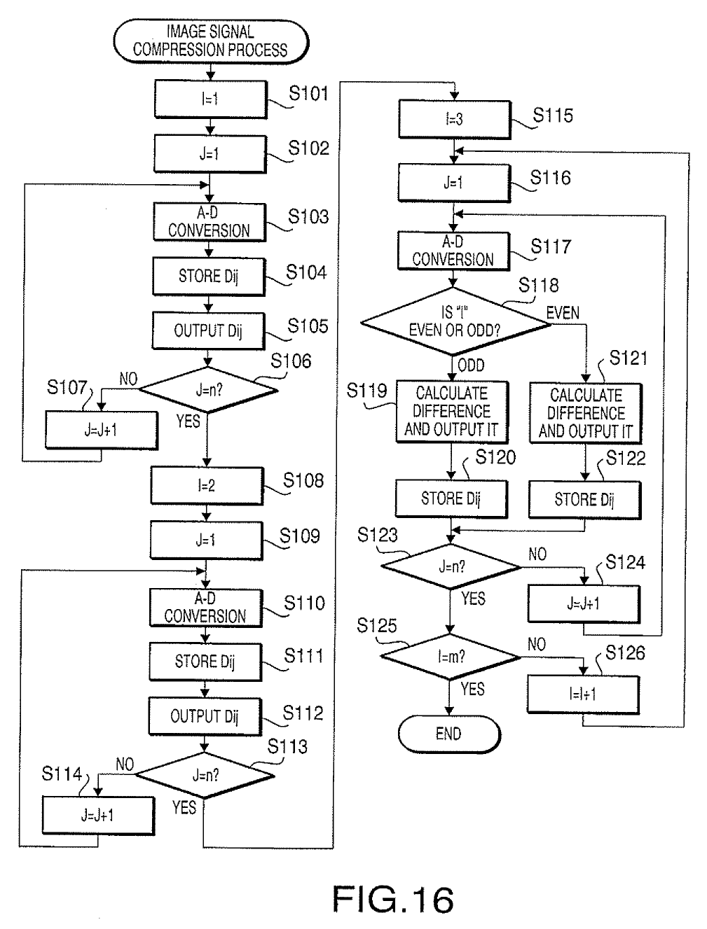

FIG. 16 is a flowchart illustrating an image signal compression process executed under control of the control unit 232y of the DST chip 230y.

First, the control unit 232y executes initialization (steps S101 and S102). In step S101, 1 is assigned to “I” (line number). In step S102, 1 is assigned to “J” (column number). In this stage, the control unit 232y formats the line memories. The line number “I” (I=1, . . . , m) represents a row address of the pixel array, and “J” (J=1, . . . , n) represents a column address of the pixel array. When the image signal received by the antenna 234 is inputted to the A-D converter 236, the control unit 232y controls the A-D converter 236 to convert the analog pixel signal of the pixel (I, J) to a digital signal D_{IJ}(step S103).

Then, the control unit 232y stores the digital signal Du at an address j (j=1, . . . , n) in the line memory 240a (j=1 when a first column is processed). Then, the control unit 232y outputs the digital signal D_{IJ} to the interface 242 (step S105). Next, in step S106, the control unit 232y judges whether the column number J is equal to n. If the column number J is equal to n (S106: YES), i.e., if steps S103 to S105 are executed for all the pixels in the first line, control proceeds to step S108. If the column number J is not equal to n (S106: NO), i.e., if at least one of the pixel signals in the first line is not subjected to steps S103 to S105, the control unit 232y increments the column number J by one (step S107). Then, control returns to step S103. By executing repeatedly steps S103 and S104, the pixel signal of the first column is stored in the address 1 of the line memory 240a, the pixel signal of the second column is stored in the address 2 of the line memory 240a, and the pixel signal of the n-th column is stored in the address n of the line memory 240a.

Next, the control unit 232y sets the line number I for 2, and the column number J for 1 (steps S108 and S109). Then, the control unit 232y controls the A-D converter 236 to converts the analog signal of the pixel (2, J) to the digital signal D_{IJ} (step S110). Next, the control unit 232y stores the digital signal D_{IJ} at an address j in the line memory 240b (j=1 when a first column is processed) (step S111), and outputs the digital signal D_{IJ} to the interface 242 (step S112).

In step S113, the control unit 232y judges whether the column number J is equal to n. If the column number J is equal to n (S113: YES), i.e., if steps S110 to S112 are executed for all the pixel signals in the second line, control proceeds to step S115. If the column number J is not equal to n (S113: NO), i.e., if at least one of the pixel signals in the second line is not subjected to steps S110 to S112, the control unit 232y increments the column number J by one (step S114). Then, control returns to step S110.

In step S115, the control unit 232y sets the line number I for 3. Then, the control unit 232y sets the column number J for 1 (step S116). Next, the control unit 232y controls the A-D converter 236 to convert the analog signal of the pixel (I, J) to a digital signal D_{IJ} (step S117).

Next, in step S118, the control unit 232y judges whether the line number I is an odd number or an even number. If the line number I is an odd number (S118: odd), the control unit 232y executes a difference operation for the digital signal D_{IJ} stored in step S117 and the digital signal D_{IJ} stored at the address J in the line memory 240a, and outputs the difference signal to the interface 242 (step S119).

Next, the control unit 232y stores the digital signal Du converted in step S117 at the address J in the line memory 240a (step S120). That is, the difference signal between the pixel signal of the pixel at a column number in the line I and the pixel signal of the pixel at the same column in the line (I-2) previously stored in the line memory 240a is obtained. For example, if the digital signal D_{IJ} converted in step S117 is the pixel signal of the pixel (5,8), the line number I is an add number (“5”) and the column number J is (“8”). In this case, the difference operation is executed for the current digital signal Du and the digital signal D_{IJ} (i.e., the pixel signal of the pixel (3,8)) stored at the address 8 in the line memory 240a, and the obtained difference signal is outputted to the interface 242. Next, the control unit 232y writes the digital signal D_{IJ} converted in step S117 in the line memory 249a at the address 8. As described above, since these neighboring same color pixels have strong correlation with each other, the output values of the difference signals take low values as shown in FIG. 15C,

If the line number I is an even number (S118: even), the control unit 232y executes the difference operation for the current digital signal D_{IJ} and the digital signal D_{IJ} stored at the address J in the line memory 240b, and outputs the obtained difference signal to the interface 242 (step S121). For example, if the digital signal D_{IJ} converted in step S117 is the pixel signal of the pixel (10,15), the line number I is an even number (“10”) and the column number J is (“15”). In this case, the difference operation is executed for the current digital signal D_{IJ} and the digital signal D_{IJ} (i.e., the signal of the pixel (8,15)) stored at the address 15 in the line memory 240b, and the obtained difference signal is outputted to the interface 242. Next, the control unit 232y writes the digital signal Du converted in step S117 in the line memory 240b at the address 15. As described above, since these neighboring same color pixels have strong correlation with each other, the output values of the difference signals take low values as shown in FIG. 15C.

As described above, according to the embodiment, the pixel signals are separated from the image signal for each of the lines having the same color arrangement, and the pixel signals separated line by line are stored in the respective line memories. That is, each line having R and G pixel signals are stored in the line memory 240a, while each line having G and B pixel signals are stored in the line memory 240b.

After step S120 or S122 is processed, the control unit 232y judges whether the column number J is equal to n (step S123). If the column number J is equal to n (S123: YES), i.e., steps S118 to S122 are finished for all the pixel signals in the current line, control proceeds to step S125. If the column number J is not equal to n (S123: NO), i.e., there is a pixel signal not subjected to steps S118 to S122 in the current line, the control unit 232y increments the column number J by one (step S124). Then, control returns to step S117.

In step S125, the control unit 232y judges whether the line number I is equal to m. If the line number I is equal to m (S125: YES), the image signal compression process terminates because in this case all the pixel signals have been processed. If the column number I is not equal to m (S125: NO), the control unit 232y increments the line number I by one because in this case there is a line not subjected to the image signal compression process (step S126). Then, control returns to step S116 to process the next line.

In this embodiment, a parameter of a pixel to be used to distinguish one of the two line memories from the other is information on whether a digital signal represents a pixel for an even line or an odd line. That is, there is no necessity to use a parameter to distinguish a pixel in a given color in a line from another pixel in another color in the same line.

### Fourth Embodiment

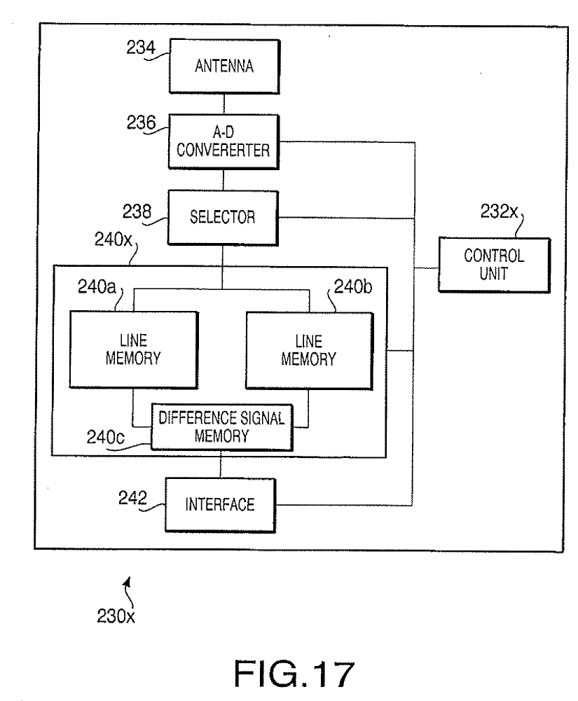

Hereafter, an endoscope system according to a fourth embodiment is described. Since a general configuration of the endoscope system according to the fourth embodiment is substantially the same as that of the third embodiment, the drawings of the first and third embodiments are also used to explain the endoscope system according to the fourth embodiment. In this embodiment, DST chips 230x(see FIG. 17) are distributed over the diagnostic jacket 200. In this embodiment, to elements, which are substantially the same as those of the first embodiment, the same reference numbers are assigned, and explanations thereof will not be repeated.

FIG. 17 is a block diagram of the DST chip 230x. As shown in FIG. 17, the DST chip 230x includes a control unit 232x, the antenna 234, the A-D converter 236, the selector 238, a memory 240X, and the interface 242. The memory 240X includes a difference signal memory 240c and the line memories 240a and 240b. The difference signal memory 240c stores difference signals generated in an image signal compression process described below, The difference signal memory has a memory size enough to store a line of difference signals.

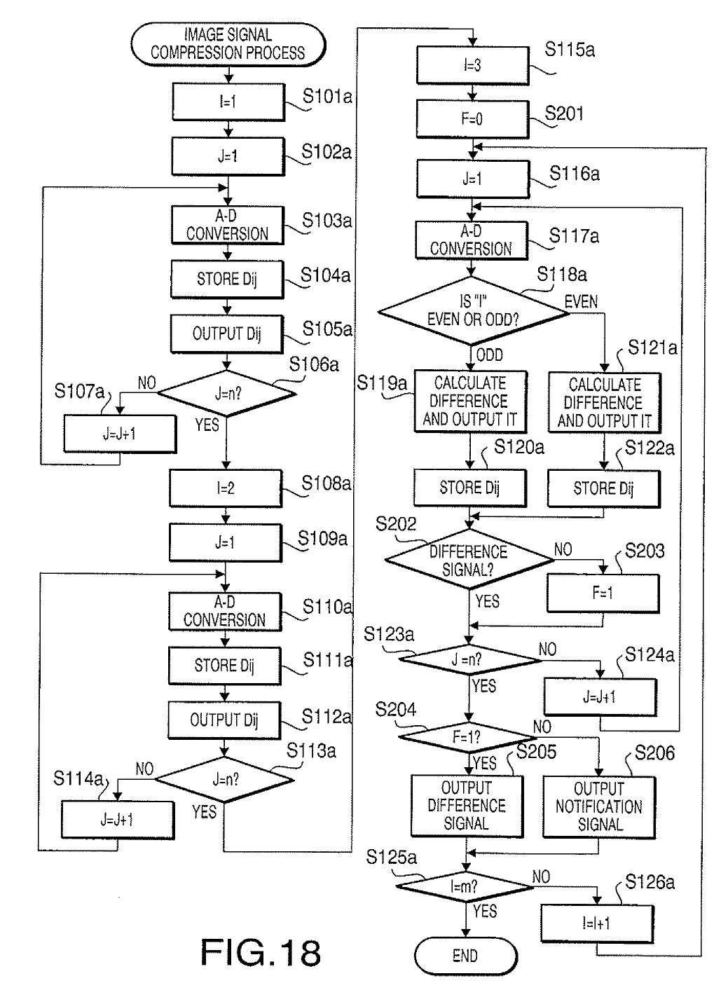

FIG. 18 is a flowchart illustrating the image signal compression process according to the fourth embodiment. It should be noted that the image signal compression process according to the fourth embodiment is configured as a variation of the image signal compression process according to the third embodiment shown in FIG. 16.

First, the control unit 232x executes steps S101a to S115a (which are substantially the same as steps S101 to S115). Then, the control unit 232x sets a flag F to zero (step S201). The flag F is used to indicate whether there is a difference between output of the image signal of the current line and output of the image signal of a line two lines ahead of the current line (i.e., between outputs of the neighboring lines having the same color arrangement). If the flag F is 0, the output values of the pixel signals in one of the targeted two lines are equal to output values of the pixel signals in the other of the targeted two lines. If the flag F is 1, at least one of the output values of the pixel signal in one of the targeted two lines is different from the output value of the corresponding pixel signal in the other line of the targeted two lines.

Next, the control unit 232x executes steps S116a to S122a (which are substantially the same as steps S116 to S122 in the image signal compression process according to the third embodiment). En this embodiment, the difference signal obtained in step S 119a or S121a is not outputted to the interface 242, but is outputted to the difference signal memory 240c. The difference signals corresponding to the pixels signals are stored at respective addresses in the difference signal memory 240c (i.e., difference signal of the first column is stored at address “1”, difference signal of the second column is stored at address “2”, . . . and the difference signal of the n-th column is stored at address “n”). In this storing process, the control unit 232x overwrites pixel signals of a previous line with pixel signals of the current line.

In step S202, the control unit 232x judges whether the output value of the difference signal obtained in step S119a or S121a is “0” (i.e., whether the output value of the current pixel signal and the output value of the corresponding pixel signal in a line two lines ahead of the current line are equal to each other). If the output values of these pixel signals are not equal to each other (S202: NO), the control unit 232x sets the flag F to “1” (step S203). Then, control proceeds to step S123a, If the output values of these pixel signals are equal to each other (S202: YES), control proceeds to step S123a without changing the flag F.

In step S123a, the control unit 232x judges whether the column number J is equal to “n”. If the column number J is equal to “n” (S123a: YES), control proceeds to step S204. If the column number J is not equal to “n” (S123a: NO), the control unit 232x increments the column number J by one (step S124a). Then, control returns to step S117a.

In step S204, the control unit 232x judges whether the flag F is “1”. If the flag F is “1” (S204: YES), the control unit 232x judges that at least one of the output values of the pixel signals in one of the targeted two lines is different from the output value of the corresponding pixel signal in the other of the targeted two lines. In this case, the control unit 232x outputs a line of difference signals stored in the difference signal memory 240c to the interface 242 so that the difference signals are transmitted another DST chip (step S205). Then, the control unit 232x executes steps S125a and S126a which are substantially the same as steps S125 and S126 in the image signal compression process shown in FIG. 16.

If the flag F is “0” (S204: NO), the control unit 232x judges that the output values of the pixel signals in one of the targeted two lines are equal to the output values of the corresponding pixel signals in the other of the targeted two lines (i.e., there is no difference between the image signals of the targeted two lines). In this case, the control unit 232x outputs a notification signal indicating that output vales of the pixel signals in the targeted two lines are equal to each other, to the interface 242 (step S206). Then, the control unit 232x executes steps S125a and S126a.

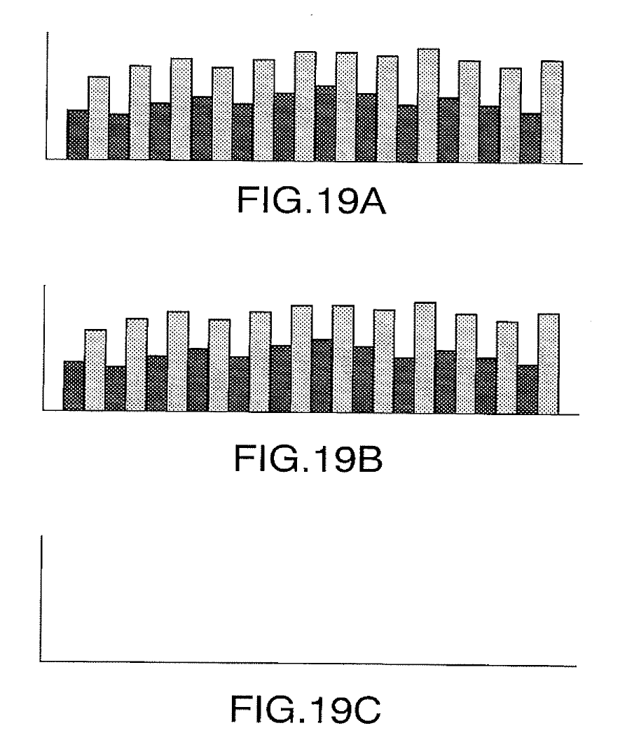

FIGS. 19A to 19C are explanatory illustrations for explaining the case where the output values of the pixel signals in the targeted two lines are equal to each other. In each of FIGS. 19A to 19C, the vertical line represents an output level of a pixel signal, and the horizontal line represents pixels arranged in a horizontal direction on the solid-state image pickup device 112. FIG. 19A illustrates output values of pixel signals in an (i-2)-th line. FIG. 19B illustrates output values of pixel signals in an i-th line (i.e., the current line). FIG. 19C illustrates output values of difference signals representing differences between output values of the (i-2)-th line and output values of the i-th line. Since the output values of the (i-2)-th line and the output values of the i-th line are equal to each other, all the output values of the difference signal are substantial zero as shown in FIG. 19C.

It should be noted that the notification signal does not represent information concerning a line of difference signals but represents the information indicating that output vales of the pixel signals in the targeted two lines are equal to each other. Therefore, the data amount of the notification signal is smaller than that of the difference signals corresponding to one line. As a result, network traffic in the signal channel formed between the DST chips can be reduced, and efficiency of signal transmission between the DST chips can be enhanced.

Although the present invention has been described in considerable detail with reference to certain preferred embodiments thereof, other embodiments are possible.

In the above mentioned embodiment, the number of pixels in a frame is continuously counted so that the separated pixel signals are treated in different fashions in accordance with the counted number of pixels. However, by obtaining a pixel address (row and column addresses) of each pixel, it is possible to appropriately separate each pixel signal from the image signal in accordance with the pixel address. The identification information to be assigned to a reference signal or a difference signal may be information representing a pixel address of each pixel signal.

In the above mentioned embodiment, each memory has two memory segments. However, each memory may be configured to have more than two memory segments. For example, the memory 240R may be provided with (n/2) memory segments. In this case, the DST chip can execute the image compression process more quickly while storing all the R-pixel signals of one line in the memory 240R without executing a copying operation as show in step S14 of FIG. 6. That is, the image signal compression process can be simplified.

In step S15 of the above mentioned embodiment, the control unit judges whether a sequence of steps for one line is finished in accordance with the count C_{P}. However, such judgment may be conducted according to whether a horizontal synchronization signal H is read.

In step S16 of the above mentioned embodiment, the control unit judges whether a sequence of steps for one frame is finished in accordance with the count C_{H}. However, such judgment may be conducted according to whether a vertical synchronization signal V is read.

In the above mentioned embodiment, G-pixel signals in an odd line and G-pixel signals in an even line are stored in different memories (memories 240G1 and 240G2). However, a single memory for G-pixels may be used in place of the two memories 240G1 and 24002 because the signal separation processes for even and odd lines are not concurrently processed.

In the above mentioned embodiment, the R-pixel signals, G-pixel signals in an odd line, G-pixel signals in an even line, and B-pixel signals are stored in different four memories, respectively. However, the image signal compression process may be performed by two different memories respectively storing pixels in an odd line and pixels in an even line.

In the above mentioned embodiment, the image signal compression is performed for the image signal generated by the solid-state image pickup device 112 having a primary color filter. However, an image signal generated by an image pickup device having a complementary color filer (e.g., a YMCG (Yellow, Magenta, Cyan, Green) filter) may be processed.

In the above mentioned second embodiment, four DST chips are selected as signal reception chips. However, the image signal compression process may be executed using more than four DST chips selected as signal reception chips. By increasing the number of signal reception chips, processing burden on each chip can be reduced.

In the above mentioned third embodiment, a line of pixel signals are stored in each line memory. However, two lines of pixel signals (or more than two lines of pixel signals) may be stored in each line memory, In this case, two neighboring lines having the same color arrangement are stored in each line memory. Specifically, pixel signals of neighboring odd lines (e.g., fifth and seventh lines) are stored in the line memory 240a, and pixel signals of neighboring even lines (e.g., sixth and eighth lines) are stored in the line memory 240b. The control unit 232y executes the difference operation process for each of pixel signals having the same column address in each line memory. In this case, data of the image signal can be effectively compressed and the compressed signal is outputted to the interface 242. When pixel signals of a new line are obtained, the control unit 232y may overwrite pixel signals in one of lines having smaller values in the line memory with the pixel signals of the new line.

This application claims priority of Japanese Patent Application No. P2005-341758, filed on Nov. 28, 2005. The entire subject matter of the application is incorporated herein by reference.

What is claimed is:

1. A method of compressing an image signal including a plurality of pixel signals outputted from a pixel array in which color pixels are arranged in a predetermined array, comprising:
calculating a difference between pixel signals of neighboring same color pixels in the pixel array in accordance with predetermined order where the plurality of pixel signals are outputted from the pixel array; and
generating a difference signal representing the calculated difference between the pixel signals of neighboring same color pixels.

2. The method according to claim 1, wherein the calculating the difference is performed for each of the pixel signals outputted from the pixel array line by line.

3. The method according to claim 1, further comprising:
adding identification information concerning the difference signal to the difference signal.

4. The method according to claim 1, wherein the pixel signals are outputted from the pixel array in the predetermined order such that colors of neighboring pixel signals successively outputted are different from each other.

5. The method according to claim 1, wherein the predetermined array includes a Bayer, array.

6. The method according to claim 1, wherein the calculating the difference and the generating the difference signal are repeated so that difference signals are generated for all of the plurality of pixel signals of the pixel array.

7. A method of compressing an image signal including a plurality of pixel signals outputted from a pixel array in which color pixels are arranged in a predetermined array, comprising:
separating each of the plurality of pixel signals from the image signal in accordance with color;
storing the separated pixel signals in accordance with color;
calculating a difference between pixel signals of neighboring same color pixels using the stored pixel signals; and
generating a difference signal representing the calculated difference between the pixel signals of neighboring same color pixels.

8. A method of compressing an image signal including a plurality of pixel signals outputted from a pixel array in which color pixels are arranged in a predetermined array, comprising:
separating the plurality of pixel signals from the image signal line by line;
storing at least one line of separated pixel signals;
calculating a difference between pixel signals in neighboring lines having a same color pixel arrangement using the stored at least one line of separated pixel signals; and
generating a difference signal representing the calculated difference between the pixel signals of neighboring same color pixels.

9. A method of compressing an image signal including a plurality of pixel signals outputted from a pixel array in which color pixels are arranged in a predetermined array, comprising:
separating the plurality of pixel signals from the image signal line by line;
storing at least one line of separated pixel signals;
calculating a difference between pixel signals in a neighboring lines having a same color arrangement using the stored at least one line of separated pixel signals; and
generating a difference signal representing the calculated difference if at least one of difference signals calculated for all the pixel signals in the stored at least one line of separated pixel signals is not zero; and
generating a notification signal if all the difference signals calculated for all the pixel signals in the stored at least one line of separated pixel signals are zero.

10. The method according to claim 9, wherein the notification signal indicates that all of the difference signals calculated for all the pixel signals in the stored at least one line of separated pixel signals are zero.

11. A device for compressing an image signal including a plurality of pixel signals outputted from a pixel array in which color pixels are arranged in a predetermined array, comprising:
a signal obtaining unit configured to obtain the image signal;
a signal separation unit configured to separate each of the plurality of pixel signals from the image signal in accordance with color;
a plurality of memories respectively storing different color pixel signals separated by the signal separation unit in accordance with color; and
a difference signal generation unit configured to calculate a difference between pixel signals of neighboring same color pixels using the pixel signals stored in at least one of the plurality of memories and to generate a difference signal representing the difference.

12. The device according to claim 11, wherein each of the memories is able to store at least two neighboring pixel signals in the image signal.

13. The device according to claim 11, wherein the difference signal generation unit calculates the difference between pixel signals of neighboring same color pixels for all the plurality of pixel signals in the pixel array line by line.

14. The device according to claim 11, wherein the difference signal generation unit adds identification information concerning the difference signal to the difference signal.

15. The device according to claim 11, wherein:
the pixel array includes a Bayer array; and
the plurality of memories include a first memory storing pixel signals for red pixels in an odd line in the pixel array, a second memory storing pixel signals for green pixels in an odd line in the pixel array, a third memory storing pixel signals for green pixels in an even line in the pixel array, and a fourth memory storing pixel signals for blue pixels in an even line in the pixel array.

16. The device according to claim 11, wherein:
the pixel array includes a Bayer array; and
the plurality of memories include a first memory storing pixel signals for red pixels in an odd line in the pixel array, a second memory storing pixel signals for green pixels in odd and even lines in the pixel array, and a third memory storing pixel signals for blue pixels in an even line in the pixel array.

17. A device for compressing an image signal including a plurality of pixel signals outputted from a pixel array in which color pixels are arranged in a predetermined array, comprising:
a plurality of signal processing units respectively corresponding to different colors of pixels in the pixel array,
wherein each of the plurality of signal processing units comprises:
a signal obtaining unit configured to obtain the image signal;
a signal extraction unit configured to extract pixel signals having a predetermined color from the image signal; and
a difference signal generation unit configured to calculate a difference between pixel signals of neighboring same color pixels using the pixel signals extracted by the signal extraction unit and to generate a difference signal representing the difference.

18. A device for compressing an image signal including a plurality of pixel signals outputted from a pixel array in which color pixels are arranged in a predetermined array, comprising:
a signal obtaining unit configured to obtain the image signal;
a signal separation unit configured to separate the plurality of pixel signals from the image signal line by line;
a plurality of memories respectively storing different lines of pixel signals separated by the signal separation unit; and
a difference signal generation unit configured to calculate a difference between pixel signals in neighboring lines having a same color pixel arrangement using the pixel signals stored in at least one of the plurality of memories and to generate a difference signal representing the difference.

19. The device according to claim 18, wherein each of the plurality of memories is able to store a line of pixel signals.

20. The device according to claim 18, wherein
the pixel array includes a Bayer array; and
the plurality of memories includes a first line memory storing pixel signals in an odd line in the pixel array and a second line memory storing pixel signals in an even line in the pixel array.

21. A device for compressing an image signal including a plurality of pixel signals outputted from a pixel array in which color pixels are arranged in a predetermined array, comprising:
a signal obtaining unit configured to obtain the image signal;
a signal separation unit configured to separate the plurality of pixel signals from the image signal line by line;
a plurality of memories respectively storing different lines of pixel signals separated by the signal separation unit;
a difference calculation unit configured to calculate a difference between pixel signals in neighboring lines having a same color pixel arrangement the pixel signals stored in at least one of the plurality of memories; and
a difference signal generation unit configured to generate a difference signal representing the calculated difference if at least one of difference signals calculated for all the pixel signals of a line in the pixel array is not zero, and to generate a notification signal if all difference signals calculated for all the pixel signals in a line in the pixel array are zero.

22. The device according to claim 21, wherein each of the plurality of memories is able to store a line of pixel signals.

23. The device according to claim 21, wherein
the pixel array includes a Bayer array; and
the plurality of memories includes a first line memory storing pixel signals in an odd line in the pixel array and a second line memory storing pixel signals in an even line in the pixel array.

24. A wearable device, comprising:
a substrate including a conductive sheet and a plurality of diffusive signal-transmission chips distributed over the conductive sheet,
wherein each of the plurality of diffusive signal-transmission chips comprises a device according to claim 11,
wherein the conductive layer is formed to be able to cover at least a part of a body of a subject,
wherein information on the body of the subject is transmitted through the substrate.

25. A wearable device, comprising:
a substrate including a conductive sheet and a plurality of diffusive signal-transmission chips distributed over the conductive sheet,
wherein at least parts of the plurality of diffusive signal-transmission chips form a device according to claim 17,
wherein the plurality of signal processing units are respectively provided in different ones of the at least parts of the plurality of diffusive signal-transmission chips,
wherein the conductive layer is formed to be able to cover at least a part of a body of a subject,
wherein information on the body of the subject is transmitted through the substrate.

26. A wearable device, comprising:
a substrate including a conductive sheet and a plurality of diffusive signal-transmission chips distributed over the conductive sheet,
wherein each of the plurality of diffusive signal-transmission chips comprises a device according to claim 18,
wherein the conductive layer is formed to be able to cover at least a part of a body of a subject,
wherein information on the body of the subject is transmitted through the substrate.

27. A wearable device, comprising:
a substrate including a conductive sheet and a plurality of diffusive signal-transmission chips distributed over the conductive sheet,
wherein each of the plurality of diffusive signal-transmission chips comprises a device according to claim 21,
wherein the conductive layer is formed to be able to cover at least a part of a body of a subject,
wherein information on the body of the subject is transmitted through the substrate.

28. An endoscope system, comprising:
a capsule-type endoscope having a form of a capsule; and
a wearable device according to claim 24,
wherein the capsule-type endoscope includes:
an image pickup unit configured to obtain an image of an inside of a body cavity of the subject and to generate an image signal representing the obtained image; and
a wireless communication unit configured to transmit the image signal as a radio signal,
wherein the signal obtaining unit in the wearable device includes an antenna which receives the image signal transmitted from the wireless communication unit of the capsule-type endoscope.

29. An endoscope system, comprising:
a capsule-type endoscope having a form of a capsule; and
a wearable device according to claim 25,
wherein the capsule-type endoscope includes:
an image pickup unit configured to obtain an image of an inside of a body cavity of the subject and to generate an image signal representing the obtained image; and
a wireless communication unit configured to transmit the image signal as a radio signal,
wherein the signal obtaining unit in the wearable device includes an antenna which receives the image signal transmitted from the wireless communication unit of the capsule-type endoscope.

30. An endoscope system, comprising:
a capsule-type endoscope having a form of a capsule; and
a wearable device according to claim 26,
wherein the capsule-type endoscope includes:
an image pickup unit configured to obtain an image of an inside of a body cavity of the subject and to generate an image signal representing the obtained image; and
a wireless communication unit configured to transmit the image signal as a radio signal,
wherein the signal obtaining unit in the wearable device includes an antenna which receives the image signal transmitted from the wireless communication unit of the capsule-type endoscope.

31. An endoscope system, comprising:
a capsule-type endoscope having a form of a capsule; and
a wearable device according to claim 27,
wherein the capsule-type endoscope includes:
an image pickup unit configured to obtain an image of an inside of a body cavity of the subject and to generate an image signal representing the obtained image; and
a wireless communication unit configured to transmit the image signal as a radio signal,
wherein the signal obtaining unit in the wearable device includes an antenna which receives the image signal transmitted from the wireless communication unit of the capsule-type endoscope.
